(19) United States
(12) Patent Application Publication
(10) Pub. No.: US 2007/0235038 A1
(43) Pub. Date: Oct. 11, 2007

(54) Surgical system

(75) Inventors: Red Alinsod, Laguna Beach, CA (US); Marc D. Gelnett, Houston, TX (US); James M. Fowler, Houston, TX (US)

Correspondence Address: VINSON & ELKINS L.L.P., 1001 FANNIN STREET, 2300 FIRST CITY TOWER, HOUSTON, TX, 77002-6760, US

(73) Assignee: Lone Star Medical Products, Inc., Stafford, TX (US)

(21) Appl. No.: 11/508,534

(22) Filed: Aug. 23, 2006

Related U.S. Application Data
(63) Continuation-in-part of application No. 11/401,772, filed on Apr. 11, 2006.

(51) Int. Cl. A61B 19/00

(52) U.S. Cl. 128/849

(57) ABSTRACT

The present invention provides a surgical system utilizing a medical drape adapted to overlay the body of a patient. The drape of the present invention has a lower surface for engaging the patient and a fluid impervious upper surface containing at least one adhesive portion. In one embodiment, the drape of the present invention is adapted for use in conjunction with one or more unique surgical retractors. The present invention provides a series of elastic stays each having an adhesive portion capable of releasably attaching to the adhesive portion(s) of the medical drape. In this manner, the present invention allows medical personnel to gently and effectively pull back tissue in preparation for, and during, surgical procedures. Further, the elastic capability of the stays allow medical personnel to adjust the amount of tension applied to retracted tissue during surgical procedures.

### CROSS-REFERENCE TO RELATED APPLICATIONS

The present application claims priority upon a U.S. patent application entitled “Posterior Approach Retractor Ring and Attachments System,” Ser. No. 11/401,772, filed on Apr. 11, 2006.

### FIELD OF THE INVENTION

The present invention pertains to medical devices and, more particularly, to a surgical system utilizing a medical drape.

### BACKGROUND OF THE INVENTION

Medical drapes are utilized in numerous types of surgical procedures and operations. Such drapes commonly are used to cover the patient during medical procedures, exposing to the doctors and nurses only areas of the patient requiring attention. These drapes have been designed to perform a variety of functions, including isolating the procedure site, and protecting the patient such as by reducing the likelihood for infection.

During the course of a surgical procedure or operation, the surgeon opens the patient with a scalpel, forming an incision and surgical site. As the surgeon cuts deeper, the operating room staff typically holds tissue away from the operative field using retractors. Most retractors are one piece metallic implements that retract a wound in a non-yielding manner. Manipulation and movement of the surgeon as well as movement caused by the contracting muscles or tissues of the patient can result in bruising or tearing of the tissue.

More recently, elastic surgical retractor systems have come into commercial use that include elastic stays, each having an elongated elastic member that is typically a hollow length of elastic tubing. The elastic tubing provides proximal and distal end portions. The distal end portion typically carries an elongated hook constructed of wire which is placed in the distal end of the bore of hollow tubing. In many instances, a shrink wrap is placed over the hook and tubing to hold the proximal end of the wire hook firmly in position within the bore of the tubing at the distal end. The embedded portion of the wire hook member is usually re-curved or folded. This folded proximal portion of the wire hook can expand the tubing slightly, forming a vertically extended portion that defines a handle.

There remains a need for a surgical system that provides a maximized area for stable attachment of elastic stays and is capable of securely lifting and holding large tissue segments without causing undue injury to such tissue.

### SUMMARY OF THE INVENTION

Accordingly, the present invention provides a surgical system utilizing a medical drape adapted to overlay the body of a patient. The drape of the present invention has a lower surface for engaging the patient and a fluid impervious upper surface containing at least one adhesive portion. Known surgical drapes typically provide an adhesive only upon the lower surface of the drape for use in securely affixing the lower surface of the drape to the patient prior to a surgical procedure. In one embodiment, the adhesive provided upon the upper surface of the drape of the present invention may be one or more Velcro® brand strips (or areas), or other adhesive materials. In one embodiment, the drape of the present invention is adapted for use in conjunction with one or more unique surgical retractors. In one embodiment, the present invention provides a series of elastic stays each having an adhesive portion capable of releasably attaching to the adhesive portion(s) of the medical drape. In this manner, the present invention allows medical personnel to gently and effectively pull back tissue in preparation for, and during surgical procedures. The elastic capability of the elastic stays allow medical personnel to adjust the amount of tension applied to retracted tissue during surgical procedures.

The drape of the present invention may also provide one or more enclosures for receiving surgical instruments. In one embodiment, the enclosures of the present invention are composed of one or more heat resistant materials so as to allow for the insertion of warm instruments into the enclosures without adverse effects. The drape may also be equipped with a pouch designed to receive body fluids during a surgical procedure and/or a removable rectal flap designed to cover the rectal area of a patient during surgical procedures. In one embodiment, the rectal flap is equipped with one or more adhesive portions in order to allow for the releasable attachment of the flap to one or more adhesive portions of the drape.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawing; it being understood that the drawings contained herein are not necessarily drawn to scale; wherein:

FIG. 1 is a top plan view of the surgical system of one embodiment of the present invention.

FIG. 2 is a perspective view of an elastic stay of one embodiment of the present invention.

FIGS. 2A-2B are perspective views of a portion of the upper surface of the surgical drape of one embodiment of the present invention utilized in conjunction with at least one grasping member.

FIG. 2C is a perspective view of a grasping member of one embodiment utilized in conjunction with an elastic stay.

FIG. 2D is a perspective view of a grasping member of one embodiment of the present invention.

FIG. 2E is a perspective view of a grasping member of one embodiment of the present invention.

FIG. 2F is a perspective view of a grasping member of one embodiment of the present invention.

FIG. 2G is a perspective view of a grasping member of one embodiment of the present invention.

FIG. 2H is a perspective view of a grasping member of one embodiment of the present invention.

FIG. 2I is a perspective view of a grasping member of one embodiment of the present invention.

FIG. 2J is a perspective view of a grasping member of one embodiment of the present invention.

FIG. 2K is a perspective view of a grasping member of one embodiment of the present invention.

FIG. 3 is a plan view of the surgical system of one embodiment of the present invention.

FIG. 4 is a perspective view of a portion of the upper surface of the surgical drape of one embodiment of the present invention utilized in conjunction with at least one surgical flap.

FIG. 5A is a side view of the retractor plate of one embodiment of the present invention.

FIG. 5B is a perspective view of a portion of the upper surface of the surgical drape of one embodiment of the present invention utilized in conjunction with at least one retractor plate.

FIG. 6A is a perspective view of a portion of the upper surface of the surgical drape of one embodiment of the present invention utilized in conjunction with at least one retractor plate.

FIG. 6B is a close-up perspective view of a retractor plate of one embodiment of the present invention.

FIG. 6C is a close-up perspective view of a retractor plate of one embodiment shown releasably engaging grasping members of one embodiment of the present invention.

FIG. 6D is a bottom plan view of a retractor plate of one embodiment shown releasably engaging grasping members of one embodiment of the present invention.

### DETAILED DESCRIPTION OF THE INVENTION

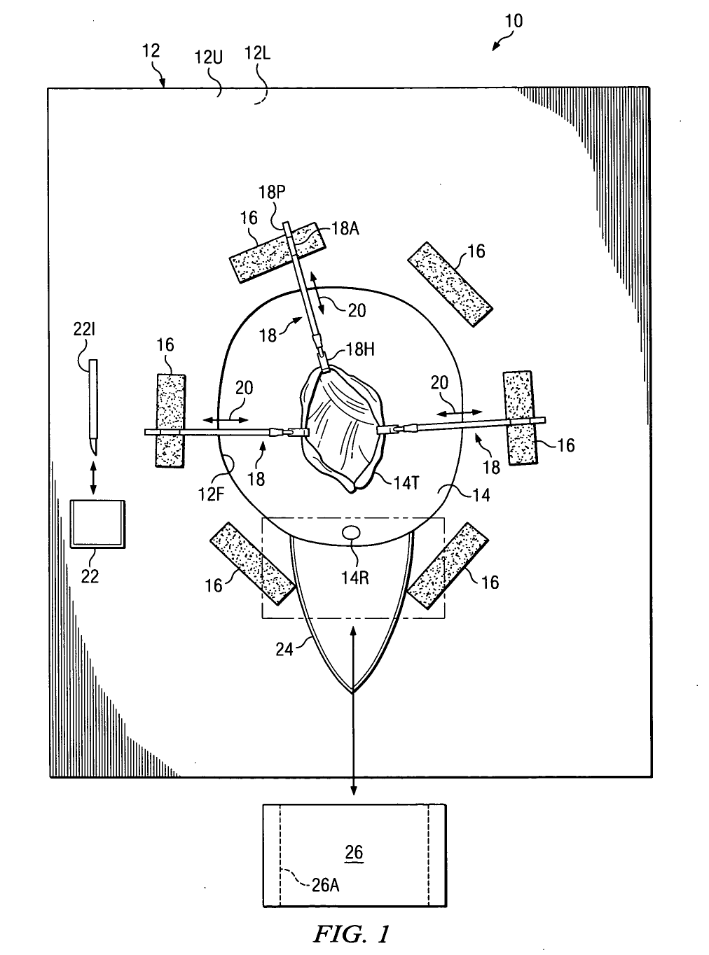

Referring to FIG. 1, the present invention is herein described as a surgical system (10) and as a surgical preparation method. The present invention provides a surgical system (10) utilizing a medical drape (12) adapted to overlay the body of a patient (14). The drape (12) of the present invention provides at least one fenestration (12F), or opening, through which medical personnel may access an area of the patient's body for surgical procedure. Typically, the drape is placed upon the body of the patient such that the fenestration is oriented to allow access to the portion of the patient's body that is undergoing surgery.

At least a portion of the drape (12) of the present invention may be composed of one or more fluid impervious materials designed to deflect fluids from the surgical workspace as well as from the body of the patient before, during, and after a surgical procedure. A portion of the drape may be composed of non-woven fabrics such as polyester-wood pulp hydro-entangled non-woven fabric treated with fluorocarbons to enhance repellency, such as Fabric 450® from Johnson & Johnson Medical, Inc., and SONTARA® from Dupont, and Tiburon™ from Ahlstrom.

The drape (12) of the present invention has a lower surface (12L) for engaging the skin of the patient and an upper surface (12U) containing one or more adhesive portions (16). In one embodiment, the upper surface (12U) of the drape contains a plurality of Velcro® brand adhesive strips/areas, Micro Velcro (a.k.a. Hook 830) strips/areas, or other suitable adhesive materials. Such adhesive portions (16) may be arranged upon the upper surface (12U) of the drape (12) in any suitable fashion. In one embodiment, the adhesive portions (16) are arranged in a generally circumferential configuration upon the upper surface of the drape and around the drape opening (12F) in order to provide medical personnel with multiple adhesive options prior to and during surgical procedures, as discussed further below.

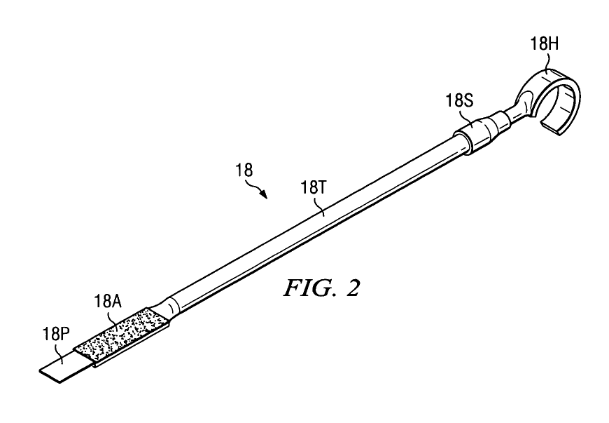

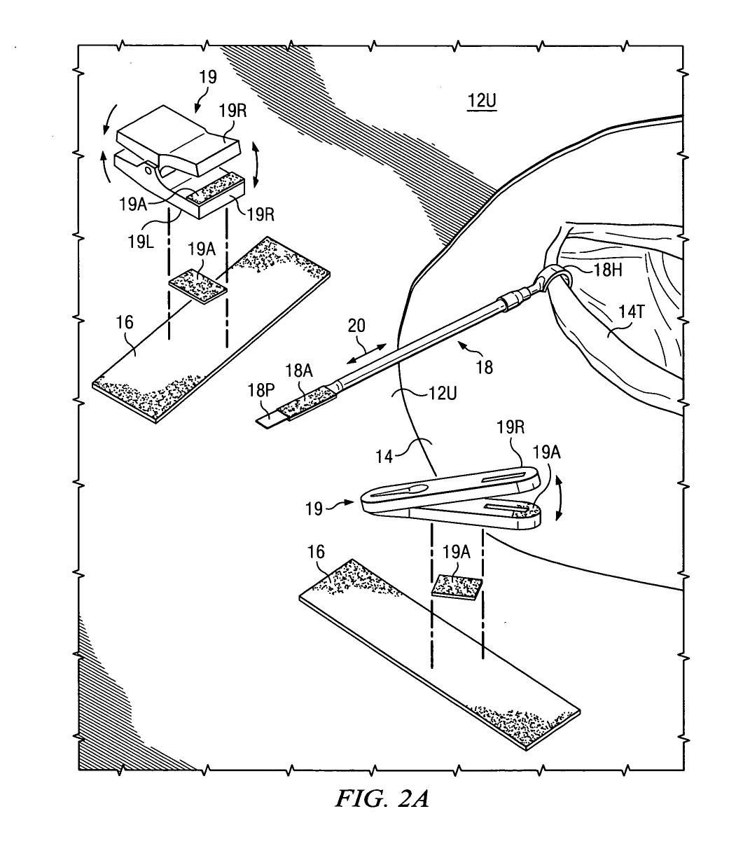

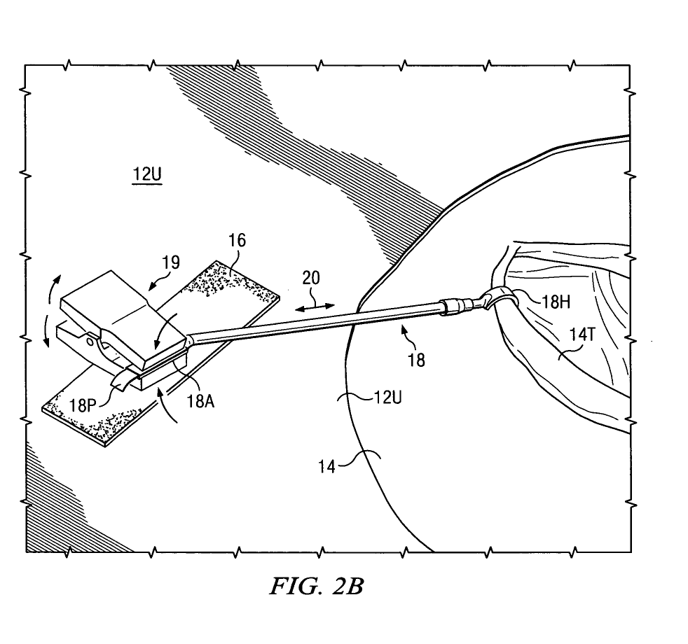

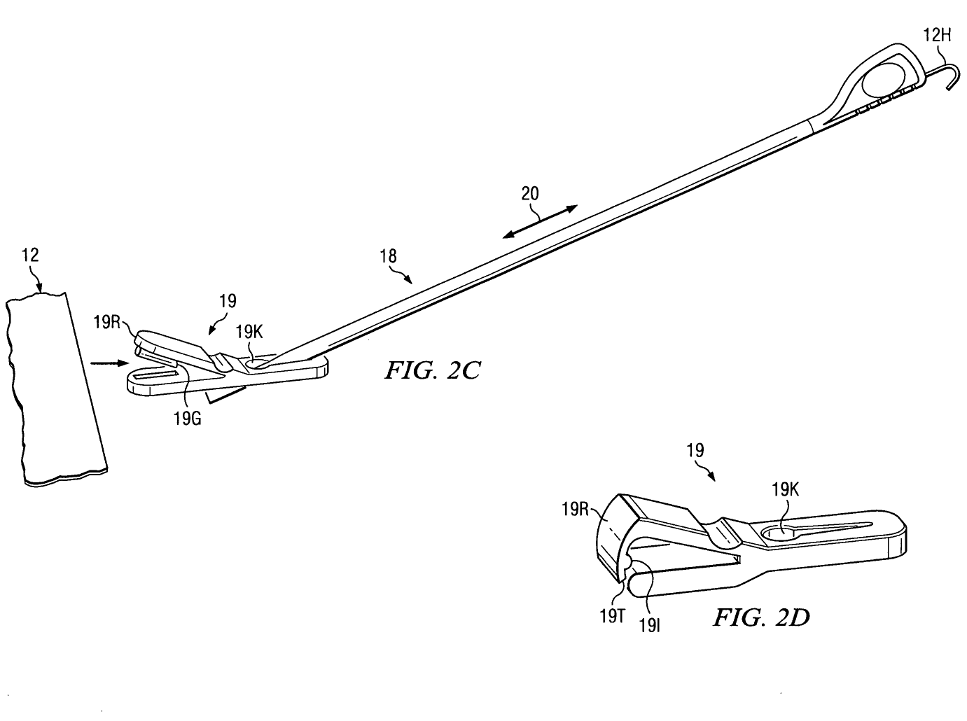

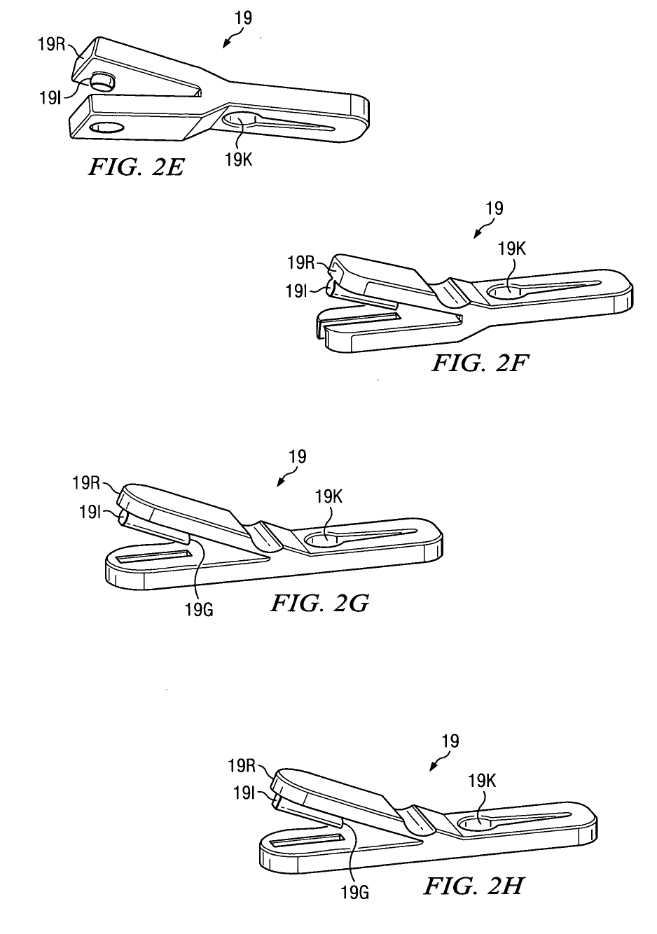

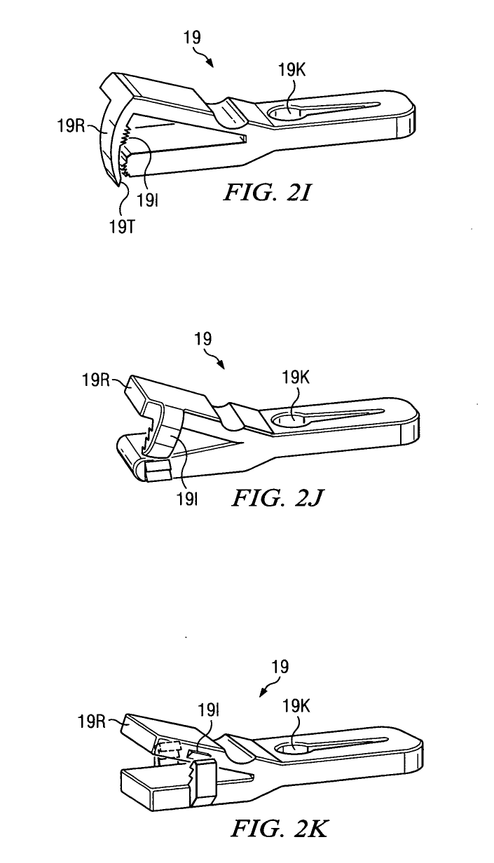

Referring to FIGS. 1-2, the drape of the present invention may be adapted for use in conjunction with one or more unique surgical retractors (18). In one embodiment, the present invention provides a series of elastic stays (18) each having an elongated elastic member that is typically a hollow length of elastic tubing (18T). The elastic tubing provides proximal and distal end portions. The distal end portion typically carries an elongated hook (18H) constructed of wire or other suitable materials which is placed in the distal end of the bore of hollow tubing. In many instances, a shrink wrap (18S) is placed over the hook and tubing to hold the proximal end of the wire hook firmly in position within the bore of the tubing at the distal end. It being understood that any known elastic stay designs, such as one piece silicone molded stays, may be utilized in conjunction with the surgical system of the present invention.

The elastic stays of the present invention may also provide an adhesive portion (18A) capable of releasably attaching to the adhesive portion(s) (16) of the medical drape (12). In one embodiment, Velcro® brand adhesive strips (or areas) are applied to the upper surface (12U) of the drape as well as to the adhesive portion (18A) of each elastic stay (18). In preparing for surgery, medical personnel need only engage tissue to be retracted (14T) with the hook portion (18H) of the elastic stay, pull back the tissue to the desired position, then engage the adhesive portion of the elastic stay (18A) with an adhesive portion (16) upon the upper surface (16U) of the drape (12). In one embodiment, each elastic stay may be equipped with a pull tab (18P) at its distal end suitable for grasping by medical personnel during elastic stay orientation and removal.

The unique configuration of the surgical system (10) provided by the present invention allows medical personnel to gently and effectively pull back tissue (14T) in preparation for, and during, surgical procedures. Further, the elastic capabilities of the elastic stays of the present invention allow medical personnel to adjust the amount of tension applied to retracted tissue during surgical procedures, as illustrated by arrow (20).

In one embodiment, the drape of the present invention also provides one or more enclosures (22) for receiving surgical instruments (22I). In one embodiment, the enclosures of the present invention are composed of one or more heat resistant materials so as to allow for the insertion of warm instruments into the enclosures without adverse effects to the patient or to the drape.

During gynecological and/or rectal procedures, body fluids may be expelled during surgery, potentially subjecting medical personnel to slippery and/or untidy conditions. As such, the drape of the present invention may also be equipped with one or more pouches (24) designed to receive body fluids during a surgical procedure. In one embodiment, the pouch of the present invention may be positioned at or below the rectal area (14R) of the patient in order to efficiently collect any such fluids.

The unique drape of the present invention may also provide a removable rectal flap (26) designed to cover the rectal area (14R) of a patient (14) during surgical procedures. In one embodiment, the rectal flap (26) is equipped with one or more adhesive portions (26A) in order to allow for the releasable attachment of the flap to one or more adhesive portions (16) of the drape. The adhesive portions of the rectal flap (26) may also utilize Velcro® brand adhesive strips (or areas) in order to allow for quick attachment to and removal from the drape. The rectal flap (26) may also be composed of a transparent adhesive, as discussed below.

The incorporation of one or more adhesive upper surfaces, such as a Velcro® releasable type of adhesive, provides a number of advantages over known drape designs. In one embodiment, the Velcro® areas pad the perimeter of the wound site to allow the surgeon to comfortably rest his or her hands against the margins of the wound during an operation while also providing a retraction attachment point for elastic stays, an attachment point for grasping member(s), an attachment point for instrument bag(s), an attachment point for one or more retractor plate arrangements, and an attachment point for one or more leg straps. These features of the present invention allows any number of instrumentalities to be conveniently placed, relocated and/or interchanged, in a cost-effective manner.

Referring to FIGS. 2A and 2B, in one embodiment, the present invention provides one or more grasping members (19) capable of engaging and retaining one or more elastic stays (18). In one embodiment, the grasping members of the present invention have one or more adhesive portions (19A). In one embodiment, the grasping member of the present invention has at least one adhesive portion on a lower surface (19L) capable of releasably attaching the grasping member to one or more adhesive portions (16) of the upper surface (12U) of the drape (12). This feature allows the surgeon or other medical personnel to releasably attach individual grasping members to an adhesive portion of the upper surface of the drape in any desired position. In one embodiment, an adhesive portion is provided upon a lower surface of the grasping member in order to allow the surgeon or other medical personnel to attach the grasping member directly to the upper surface of the drape. Further, one or more retaining portions (19R) of the grasping member (19) may be equipped with one or more adhesive portions (19A). This feature of the present invention allows the adhesive portion (19A) of the retaining portions (19R) of the grasping member (19) to securely engage an adhesive portion (18A) of one or more elastic stays (18), thus allowing for additional stability and reliability during surgical procedures.

The retaining portions of the present invention may utilize any form, type, or, number of engaging members. Such engaging members may include, but are not limited to, clamping devices that may or may not include teeth, one or more slotted section(s) capable of engaging elastic tails through compressive and/or interlocking force which further may utilize offsetting tail force vectors to promote adhesion of the grasping member(s); and/or one or more adhesives which may or may not be releasable as would be apparent to one or ordinary skill in the art in light of the disclosure herein.

The grasping member(s) of the present invention may be permanently affixed to the upper surface of the drape via any suitable mounting method and/or simply affixed to the drape via the retaining members, as described further below. Grasping members suitable for use with the present invention may include clips, hooks, clasps, or any other member capable of holding one or more elastic stays under tension. The unique configuration of the present invention allows medical personnel to gently and effectively pull back tissue (14T) in preparation for, and during, surgical procedures. Further, the elastic capabilities of the elastic stays of the present invention allow medical personnel to adjust the amount of tension applied to retracted tissue during surgical procedures.

Referring to FIGS. 2C-2K, in one embodiment, the grasping member(s) of the present invention may contain a retaining portion for mechanically interlocking with the drape or one or more elastic stays. These retaining portions may take the form of a jaw-like structure comprising one or more hinges, one or more articulating arms, and/or one or more interlocking sections. In one embodiment, the retaining member(s) of the present invention may be designed to receive and releasably secure a portion of the drape. Further, the grasping member(s) of the present invention may also provide one or more slots (19K) for receiving and securing a portion of the elastic stay(s). In one embodiment, one or more retaining members may be equipped with one or more interlocking members (19I).

In one embodiment, the interlocking members of the present invention are capable of creating an interference fit for securing the drape or one or more elastic stays to the grasping member. The interlocking members of the present invention may comprise any number of structural configurations. In one embodiment, the interlocking member may have an angled portion (19G) capable of providing increased gripping force on the drape or elastic stay as tension is increased on the opposite end of the grasping member. Such tension may also be provided by an elastic tail secured within one or more tapered slots. In one embodiment, a release tab (19T) may be provided capable of releasing the drape from the grasping member. In another embodiment, the grasping member may be permanently attached to the drape. In one embodiment, the grasping member may mechanically interlock with the tail of an elastic stay with the interlocking structure and use other methods to attach to the drape.

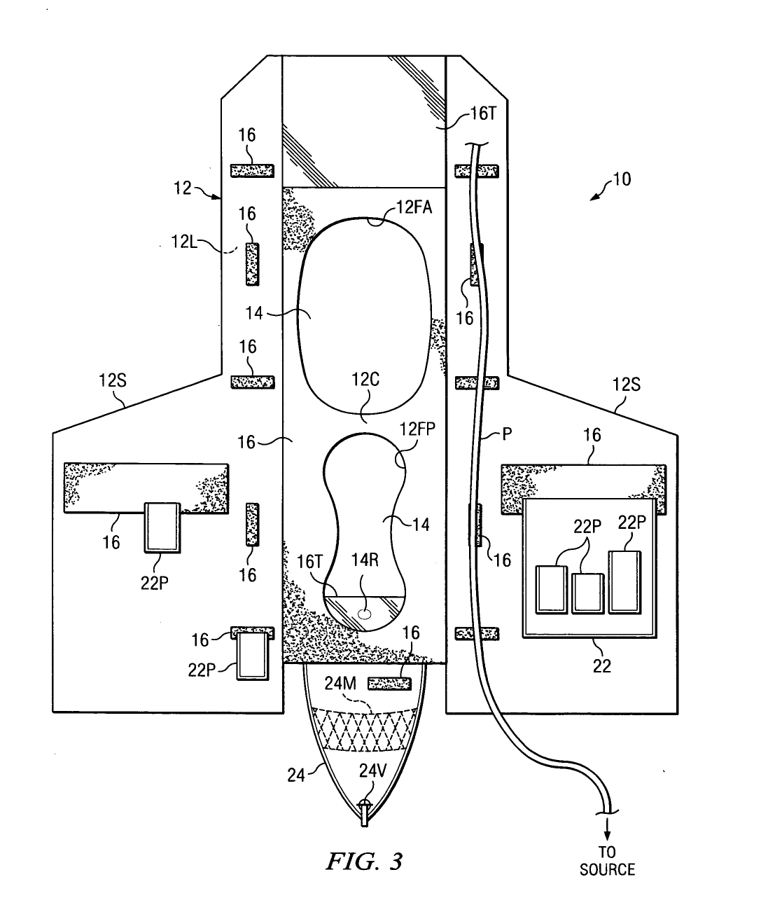

Referring to FIG. 3, in one embodiment, the present invention provides a lower fenestration (12FP) designed to provide surgical access to the posterior area of the patient. In one embodiment, the lower fenestration has an hourglass configuration designed to facilitate convenient access to the posterior area of the patient. As with the above described embodiments, the surgical drape of FIG. 3 may provide one or more adhesive areas (16) capable of retaining one or more elastic stays. For ease of illustration, elastic stays are not illustrated in FIG. 3. However, it should be readily understood that the elastic stays of the present invention may also be utilized with the surgical drape embodiment of FIG. 3.

The present invention may also utilize one or more transparent adhesives (16T). In one embodiment, at least a portion of the lower fenestration (12FP) is covered by a transparent adhesive. The transparent adhesive allows the surgeon or other medical personnel to view the patient (14) through the adhesive. This feature of the present invention is particularly useful in posterior procedures, such as the posterior intra-vaginal slingplasty (PIVS) procedure. To illustrate, many posterior procedures require an incision measured three (3) centimeters lateral and three (3) centimeters inferior to the anus (14R) of the patient. In one embodiment, a transparent adhesive is utilized upon a portion of the lower fenestration in order to allow the surgeon to make incisions using the patient's anus as a reference point.

In one embodiment, the present invention provides a transparent adhesive (16T) upon a portion of the lower fenestration. The transparent adhesive also protects the surgical area from unsanitary conditions caused by accidents arising from the patient's posterior area. It should be understood by one of ordinary skill in the art that a transparent adhesive may be applied to any number of areas upon the surgical drape of the present invention according to any number of configurations. For example, it may be useful to provide a surgical drape having a transparent portion (16T) for overlay over the head of the patient. This feature of the present invention allows the patient to look upwardly through the drape if the patient is awake, while also allowing the surgeon or other medical personnel to observe the patient's conditions should he or she be placed under anesthesia during the procedure. In one embodiment, the present invention may utilize Tegaderm® brand transparent adhesive, manufactured by 3M corporation.

In one embodiment, the surgical drape of the present invention provides an upper fenestration (12FA) designed to facilitate surgical access to the chest and/or abdominal area of the patient (14). In one embodiment, the lower fenestration (12FP) and the upper fenestration (12FA) form a center portion (12C) there-between. In one embodiment, the center portion comprises a relatively thin strip of drape material containing at least one adhesive portion (16).

In one embodiment, the upper fenestration of the present invention has an oval or generally circular configuration. Adhesive portions may be positioned upon the upper surface of the drape in any suitable fashion. In one embodiment, adhesives (16) may be positioned laterally along the sides of the drape so as to facilitate the placement and retention of various cords (P) required to operate surgical devices. This feature of the present invention allows the surgeon or other medical personnel to work without concern for “dangling” power or vacuum cords near the surgical area. The upper fenestration may be covered in Tegaderm completely or partially. In one embodiment, the Tegaderm may cover the lateral portions of the upper fenestration to allow fiducial marker identification and instrument insertion.

As described above, the surgical system of the present invention may provide one or more pockets (22) and/or sub-pockets (22P) of varying sizes for receiving medical devices and/or accessories. In one embodiment, each pocket/sub-pocket arrangement is interchangeable in that they may be attached and removed at the discretion of the surgeon. In one embodiment, this is accomplished by providing one or more adhesive areas to each pocket/sub-pocket combination. In one embodiment, adhesives may be positioned upon the side areas (12S) of the surgical drape in order to facilitate attachment and removal of any number of pocket/sub-pocket combinations. In one embodiment, the side areas (12S) of the surgical drape of the present invention are ideally suited to rest upon the patient's outstretched legs during a posterior surgical procedure.

During gynecological and/or rectal procedures, body fluids may be expelled during surgery, potentially subjecting medical personnel to slippery and/or untidy conditions. As such, the drape of the present invention may also be equipped with one or more pouches (24) designed to receive body fluids during a surgical procedure. The pouch of the present invention may be positioned at or below the rectal area (14R) of the patient or laterally along the upper fenestration edges in order to efficiently collect any such fluids. In one embodiment, the pouch provides a mesh area (24M) for retaining large particles of biological waste generated during surgical procedures. The pouch may also provide a valve (24V) or spigot through which waste products may easily be drained after surgical procedures. In one embodiment, the valve or other drainage aid may be located at the bottom portion of the pouch such that gravity assists the surgeon in draining waste fluids.

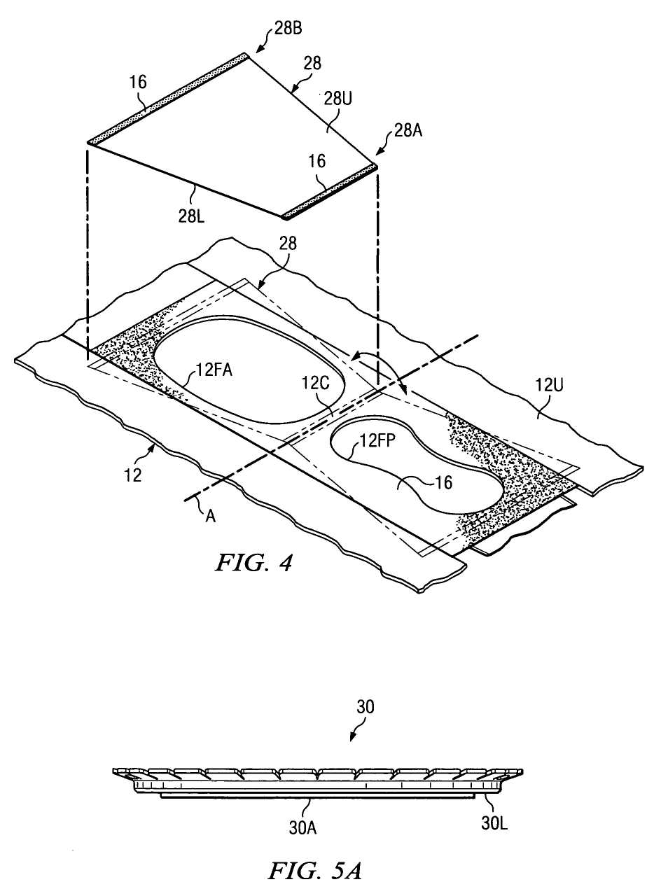

Referring to FIG. 4, the present invention may provide a surgical flap (28) capable of covering the upper fenestration or the lower fenestration. In one embodiment, the surgical flap may be equipped with adhesive at both ends (28A and 28B, respectively), such that the surgical flap may be releasably attached to one or more adhesive portions (16) upon the upper surface of the drape. In one embodiment, the surgical flap of the present invention has a trapezoidal configuration, although it would be apparent to one of ordinary skill in the art that the surgical flap could utilize any number of configurations.

In operation, a first end (28A) of the surgical flap may be releasably attached to or affixed to the center portion (12C) of the upper surface of the drape using the adhesive described above. In turn, the second end (28B) may be releasably attached or affixed to another adhesive portion of the drape such that one of the fenestrations (12FA or 12FP, respectively) is covered. In one embodiment, adhesive (16) may be positioned upon both the upper and lower surfaces (28U and 28L, respectively) of both ends (28A and 28B, respectively) of the surgical flap (28). This feature of the present invention allows the surgical flap to pivot about axis (A) in order to allow for coverage of the upper fenestration or the lower fenestration, depending on the particular needs of the surgeon. In one embodiment, the surgeon may also remove the surgical flap entirely, if not required for the surgical application at issue.

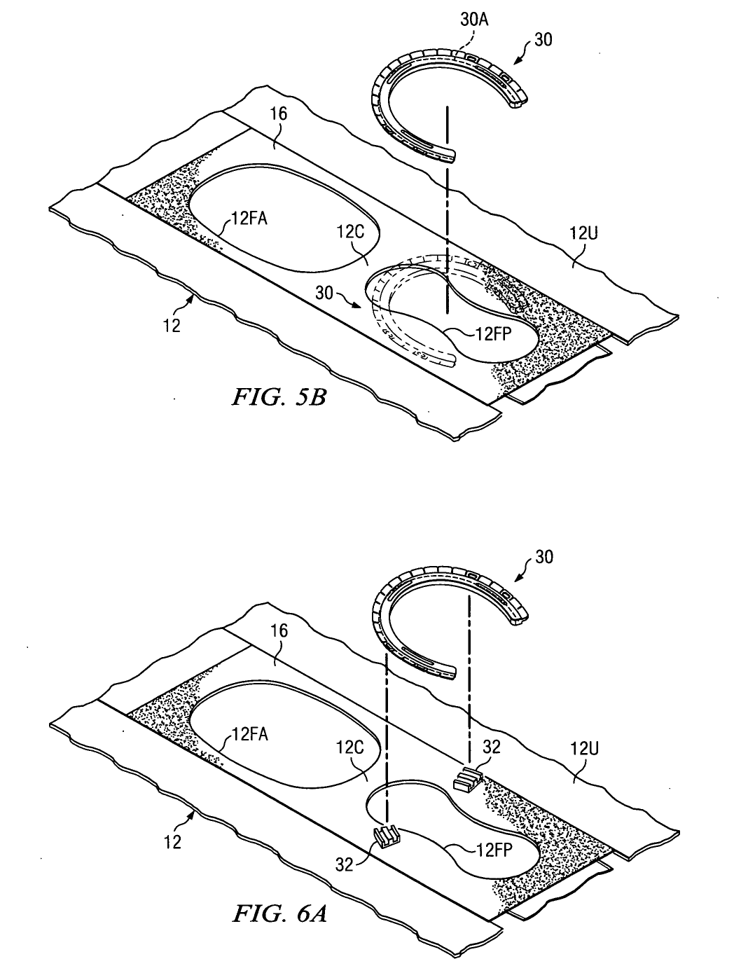

Referring to FIGS. 5A and 5B, the versatility of the surgical drape of the present invention allows it to be used with any number of surgical retractor systems. For example, retractor systems as shown and described in U.S. Pat. No. 4,274,398, issued to Frank B. Scott, Jr., on Jun. 23, 1981; U.S. Pat. No. 4,430,991, issued to W. Dale Darnell on Feb. 14, 1984, and U.S. Pat. No. 4,434,791, issued to W. Dale Darnell on Mar. 6, 1984, each being fully incorporated by reference herein, may be utilized in conjunction with the unique surgical system of the present invention. In one embodiment, the invention provides a surgical retractor plate (30) having at least one adhesive portion (30A) on its lower surface (30L). As illustrated by FIG. 5, the retractor plate of the present invention may be releasably attached to the upper surface (12U) of the surgical drape (12) of the present invention. To accomplish this, the surgeon need only place the lower surface (30L) of the retractor plate (30) firmly on the upper surface (12U) of the surgical drape (12) such that the adhesive (30A) positioned upon the lower surface (30L) of the retractor plate engages the adhesive (16) upon the upper surface (12U) of the surgical drape (12). In this manner, the present invention allows the surgeon to securely position the retractor plate upon the surgical drape. Once positioned, the surgeon or other medical personnel may utilize one or more elastic stays in conjunction with the retractor plate in order to gently spread apart tissue in preparation for surgery.

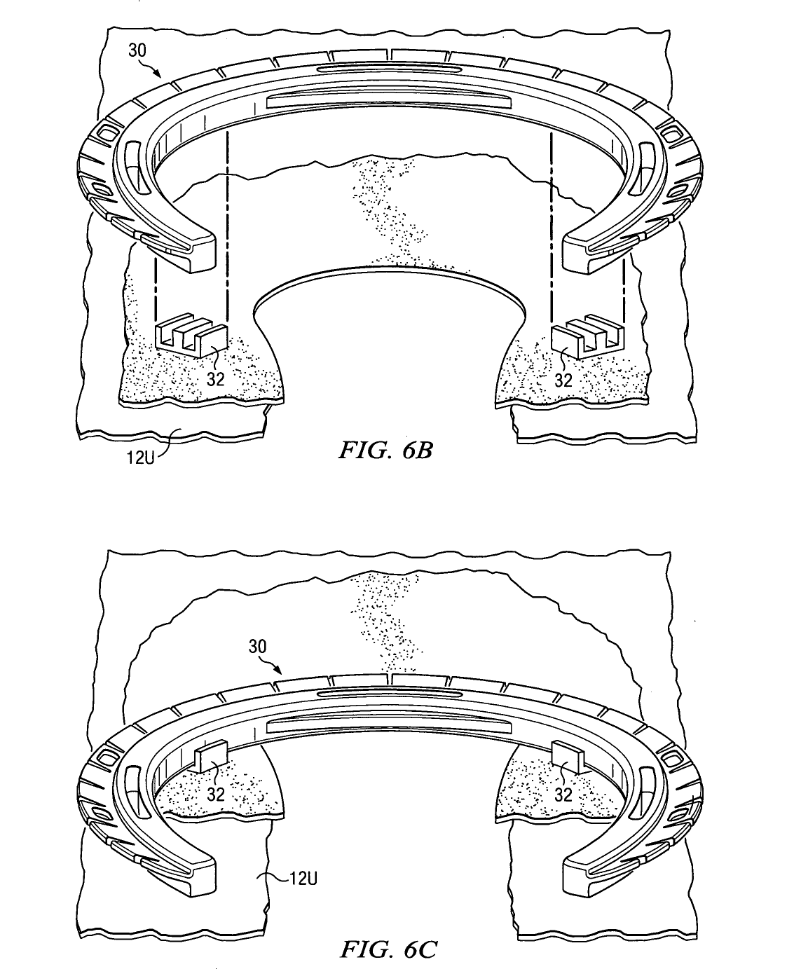

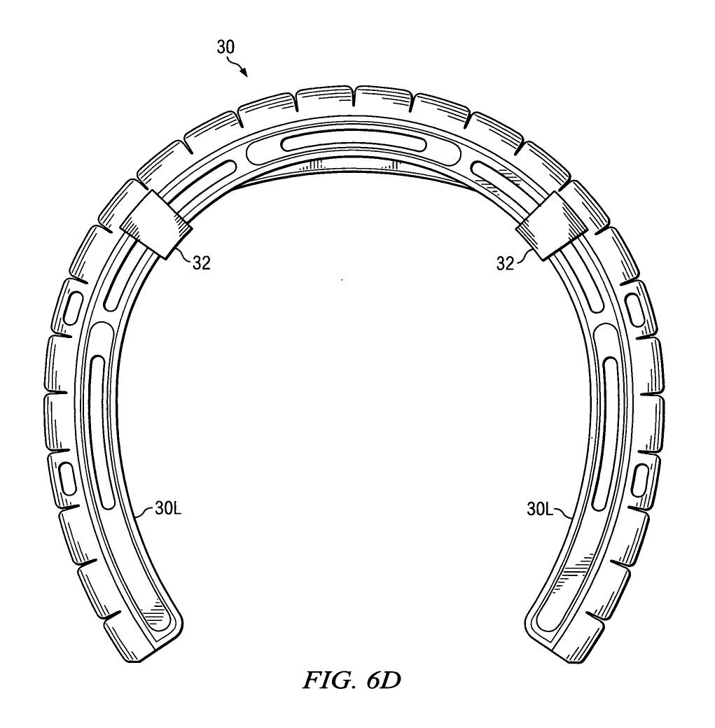

Referring to FIGS. 6A-6D, in one embodiment, the retractor plate of the present invention may utilize one or more grasping members capable of releasably attaching the plate to the drape. In one embodiment, this may involve a generally sliding motion to interlock the plate into one or more grasping members upon or within the drape. In one embodiment, a snap fit arrangement (32) may be provided such that the plate is allowed to snap into one or more receiving members upon or within the drape. The grasping member(s) upon or within the drape may be composed of any suitable rigid, semi-rigid, or soft material capable of successfully interacting with the plate. Further, such members may be constructed of material(s) that are heterogeneous or homogeneous with the drape material. This connection may also utilize one or more adhesive(s). As with the grasping members described above, snap fit arrangement (32) may be releasably or permanently attached to the upper surface of the drape. Further, it should be understood that the embodiment illustrated in FIGS. 6A-6D may be utilized with any number of surgical drape designs, whether or not the drape is equipped with adhesive (16).

It being understood that the retractor plate (30) of the present invention may comprise any number of shapes including, but not limited to, circular, triangular, or non-symmetrical configurations. The shape of the retractor plate may also be altered to correspond to the contour of the bodily region of the patient to be operated on. Further, the retractor plate of the present invention may be composed of any suitable material including semi-pliable or malleable materials. Suitable semi-pliable materials include materials that are capable of being molded into a particular shape while at the same time possessing enough resistance to shape changes to allow proper tension to be exerted on the retractor plate by an elastic stay or blade. Such materials include, but are not limited to, certain plastics and/or malleable metals such as nitinol.

Although the invention has been described with reference to specific embodiments, this description is not meant to be construed in a limited sense. Various modifications of the disclosed embodiments, as well as alternative embodiments of the invention, will become apparent to persons skilled in the art upon reference to the description of the invention. It is, therefore, contemplated that the appended claims will cover such modifications that fall within the scope of the invention.

What is claimed is:

1. A surgical system comprising:
a medical drape adapted to overlay the body of a patient, said drape having at least one fenestration through which medical personnel may access the patient's body, said drape having a lower surface for engaging the patient and an upper surface having at least one adhesive portion

2. The surgical system of claim 1, wherein at least a portion of said adhesive is positioned upon said upper surface adjacent to said fenestration.

3. The surgical system of claim 2, further comprising one or more surgical retractors adapted for use with said drape.

4. The surgical system of claim 3, wherein said surgical retractor comprises one or more elastic stays having a proximate hook portion for releasably engaging tissue.

5. The surgical system of claim 4, wherein said elastic stay further comprises at least one adhesive portion.

6. The surgical system of claim 5, wherein said adhesive portion of said elastic stay is positioned upon a distal end of said elastic stay.

7. The surgical system of claim 4, further comprising at least one grasping member adapted for use with said drape.

8. The surgical system of claim 7, wherein said grasping member further comprises one or more adhesive portions.

9. The surgical system of claim 8, wherein at least one of said adhesive portions of said grasping member is positioned upon a lower surface of said grasping member.

10. The surgical system of claim 7, wherein said grasping member further comprises at least one retaining member.

11. The surgical system of claim 10, wherein said retaining member comprises at least one adhesive portion.

12. The surgical system of claim 7, wherein said grasping member further comprises one or more slots.

13. The surgical system of claim 7, wherein said grasping member comprises a first end having at least one retaining member and a second end having one or more slots.

14. The surgical system of claim 1, wherein at least a portion of said adhesive comprises a transparent material.

15. The surgical system of claim 14, wherein at least a portion of said fenestration is covered by said transparent material.

16. The surgical system of claim 1, wherein said fenestration has a generally hourglass configuration.

17. The surgical system of claim 1, wherein said drape comprises a first fenestration and a second fenestration forming a central area there-between, said central area having at least one adhesive portion.

18. The surgical system of claim 17, further comprising a surgical flap having an adhesive portion for engaging said adhesive portion of said central area.

19. The surgical system of claim 4, wherein at least one of said surgical retractors comprise a retractor plate.

20. The surgical system of claim 19, wherein said retractor plate further comprises a lower surface containing at least one adhesive.

21. The surgical system of claim 18, wherein said surgical flap has a generally trapezoidal configuration.

22. A method of conducting a surgical procedure comprising the steps of:
providing a medical drape adapted to overlay the body of a patient, said drape having at least one fenestration through which medical personnel may access the patient's body, said drape having a lower surface for engaging the patient and an upper surface having at least one adhesive portion;
positioning the drape upon the user such that the lower surface of the drape engages the body of the patient and the fenestration is oriented to allow medical personnel to access the patient's body;
providing at least one elastic stay adapted for use with said drape, said elastic stay comprising an elongated elastic member having a proximate hook portion and a distal adhesive portion;
grasping a portion of the patient's tissue utilizing the hook portion of the surgical retractor;
retracting the patient's tissue by pulling the distal end of the elastic stay in a direction away from the patient; and
attaching the adhesive portion of the elastic stay to said adhesive portion on the upper surface of the drape.

23. The method of claim 22, wherein said upper surface of said drape further comprises at least one grasping member, said grasping member having one or more retaining members for releasably engaging said distal portion of said elastic stay.

24. The method of claim 22, further comprising the additional steps of:
providing a retractor plate having a lower surface containing one or more adhesives;
securely positioning said lower surface of said retractor plate upon said upper surface of said surgical drape adjacent to said fenestration; and
attaching the distal portion of the elastic stay to the retractor plate.

25. The method of claim 22, wherein at least a portion of said adhesive comprises a transparent material.

26. The method of claim 25, wherein at least a portion of said fenestration is covered by said transparent material.

27. The method of claim 22, wherein said fenestration has a generally hourglass configuration.

28. The method of claim 22, wherein said drape further comprises a first fenestration and a second fenestration forming a central area there-between, said central area having at least one adhesive portion.

29. The method of claim 22, further comprising the additional step of providing a surgical flap having an adhesive portion for engaging said adhesive portion of said central area.

30. The method of claim 29, further comprising the additional step of grasping at least one surgical instrument utilizing said surgical flap.

31. A surgical system comprising:
a medical drape adapted to overlay the body of a patient, said drape having at least one fenestration through which medical personnel may access the patient's body, said drape having a lower surface for engaging the patient; and
at least one grasping member adapted for use with said drape.

32. The surgical system of claim 31, wherein at least one grasping member is positioned upon an upper surface of said drape adjacent to said fenestration.

33. The surgical system of claim 31, further comprising one or more surgical retractors adapted for use with said drape.

34. The surgical system of claim 33, wherein one or more of said surgical retractors comprise elastic stays having a proximate hook portion for releasably engaging tissue and a distal portion for engaging a portion of said grasping member.

35. The surgical system of claim 34, wherein said grasping member further comprises at least one retaining member.

36. The surgical system of claim 35, wherein said retaining member is capable of releasably engaging said distal portion of said elastic stay.

37. The surgical system of claim 35, wherein said retaining member is capable of releasably engaging said upper surface of said drape.

38. The surgical system of claim 34, wherein said distal end of said elastic stay further comprises at least one adhesive portion.

39. The surgical system of claim 34, wherein said retaining member further comprises at least one adhesive portion.

40. The surgical system of claim 34, wherein said grasping member further comprises one or more slots for releasably engaging said distal end of said elastic stay.

41. The surgical system of claim 40, wherein said grasping member comprises a first end having at least one retaining member and a second end having one or more slots.

42. The surgical system of claim 31, wherein at least a portion of said fenestration is covered by a transparent material.

43. The surgical system of claim 31, wherein said fenestration has a generally hourglass configuration.

44. The surgical system of claim 31, wherein said drape comprises a first fenestration and a second fenestration forming a central area there-between, said central area having at least one adhesive portion.

45. The surgical system of claim 44, further comprising a surgical flap having an adhesive portion for engaging said adhesive portion of said central area.

46. The surgical system of claim 45, wherein said surgical flap has a generally trapezoidal configuration.

47. The surgical system of claim 44, wherein at least one of said surgical retractors comprises a retractor plate.

48. The surgical system of claim 47, wherein one or more of said grasping members are capable of releasably engaging a lower surface of said retractor plate.

49. The surgical system of claim 48, wherein one or more of said grasping members have a generally E-shaped configuration.

50. A method of conducting a surgical procedure comprising the steps of:
providing a medical drape adapted to overlay the body of a patient, said drape having at least one fenestration through which medical personnel may access the patient's body, said drape having a lower surface for engaging the patient and an upper surface providing at least one grasping member positioned adjacent to at least one fenestration;
positioning the drape upon the user such that the lower surface of the drape engages the body of the patient and the fenestration is oriented to allow medical personnel to access the patient's body;
providing at least one elastic stay adapted for use with said drape, said elastic stay comprising an elongated elastic member having a proximate hook portion and a distal adhesive portion;
grasping a portion of the patient's tissue utilizing the hook portion of the surgical retractor;
retracting the patient's tissue by pulling the distal end of the elastic stay in a direction away from the patient; and
releasably attaching the distal end of the elastic stay to at least a portion of the grasping member positioned upon the upper surface of the drape.

51. The method of claim 50, wherein one or more of said grasping members further comprise one or more retaining members.

52. The method of claim 51, wherein said distal end of said elastic stay engages at least one of said retaining members.

53. The method of claim 50, wherein one or more of said grasping members further comprise one or more slots.

54. The method of claim 53, wherein said distal end of said elastic engages at least one of said slots.

55. A method of conducting a surgical procedure comprising the steps of:
providing a medical drape adapted to overlay the body of a patient, said drape having at least one fenestration through which medical personnel may access the patient's body, said drape having a lower surface for engaging the patient and an upper surface providing at least one grasping member positioned adjacent to the fenestration;
positioning the drape upon the user such that the lower surface of the drape engages the body of the patient and the fenestration is oriented to allow medical personnel to access the patient's body;
positioning a retractor plate upon the upper surface of the drape;
providing at least one elastic stay adapted for use with said drape, said elastic stay comprising an elongated elastic member having a proximate hook portion and a distal adhesive portion;
releasably attaching a lower surface of said retractor plate to one or more of said grasping members residing upon the upper surface of the drape;
grasping a portion of the patient's tissue utilizing the hook portion of the surgical retractor;
retracting the patient's tissue by pulling the distal end of the elastic stay in a direction away from the patient; and
releasably attaching the distal end of the elastic stay to a portion of the retractor plate.

56. The method of claim 55, wherein at least a portion of said fenestration is covered by a transparent material.

57. The method of claim 55, wherein said fenestration has a generally hourglass configuration.

58. The method of claim 55, wherein at least one of said grasping members has a generally E-shaped configuration.

59. The method of claim 55, wherein said drape further comprises a first fenestration and a second fenestration forming a central area there-between, said central area having at least one adhesive portion.

60. The method of claim 59, further comprising the additional step of providing a surgical flap having an adhesive portion for engaging said adhesive portion of said central area.
